(19) United States
(12) Patent Application Publication
(10) Pub. No.: US 2013/0235795 A1
(43) Pub. Date: Sep. 12, 2013

(54) DISTRIBUTED APPLICATION SYSTEM AND METHOD FOR CONTROLLING QUALITY OF SERVICE IN DATA TRANSMISSION THEREOF

(71) Applicant: INDUSTRIAL TECHNOLOGY RESEARCH INSTITUTE, Hsinchu (TW)

(72) Inventors: Yung-Shun Huang, Taichung City (TW); Yuan-Fa Lee, Tainan City (TW)

(73) Assignee: INDUSTRIAL TECHNOLOGY RESEARCH INSTITUTE, Hsinchu (TW)

(21) Appl. No.: 13/658,823

(22) Filed: Oct. 24, 2012

(30) Foreign Application Priority Data
Mar. 6, 2012 (TW) 101107503
Jun. 22, 2012 (TW) 101122432

(51) Int. Cl. H04W 28/16

(52) U.S. Cl. 370/328

(57) ABSTRACT

A distributed application system and a method for controlling QoS in data transmission are provided. The distributed application system includes an application hosting device (AHD), a wireless sensor network (WSN) and a gateway. The AHD includes a personal health manage software module, a first application programming interface (API) module and a first communication protocol module. The gateway includes a second API module, second and third communication protocol modules. The WSN, the first and second communication protocol modules support a first communication protocol, and the third communication protocol module supports a second communication protocol. The first API module cooperates with the second API module to accomplish all of default functionalities of a personal network API. The PHD is connected to the AHD via the gateway. When the AHD or the gateway transmits a message to the WSN, the QoS control of the message is performed by setting priority and acknowledge parameters.

### CROSS-REFERENCE TO RELATED APPLICATION

This application claims the priority benefits of Taiwan application serial no. 101107503, filed on Mar. 6, 2012, and Taiwan application serial no. 101122432, filed on Jun. 22, 2012. The entirety of each of the above-mentioned patent applications is hereby incorporated by reference herein and made a part of this specification.

### TECHNICAL FIELD

The disclosure relates to a distributed application system and a method for controlling quality of service in data transmission of the distributed application system.

### BACKGROUND

Continua Health Alliance has specified short range connection technology of a personal area network interface (PAN-IF) between an application hosting device (AHD) and Bluetooth health device profile personal health devices (BT HDP PHD), and a longer range connection technology of sensor local area network interface (sensor-LAN IF) between Zigbee health care PHDs (Zigbee HC PHDs).

However, the currently certified Continua PHD mostly uses BT HDP communication protocol standard, and due to the fact that the BT HDP communication protocol standard has too short connection range with traditional Continua AHD, application fields of the Continua PHD and the Continua AHD definitely will be affected. If Continua BT HDP PHD can be connected to a remote Continua AHD through Zigbee network like Zigbee HC PHDs, and transmit physiological signals to the remote Continua AHD, the aforementioned impact may be properly dealt with.

Generally, network equipments supporting different communication protocols cannot perform communications between each other. Most common approach to overcome the different protocols may be allocating a gateway between the network equipments using different communication protocols, and the gateway may be responsible for conversion of packets supporting different communication protocols. It will be a direction of research and development in this field to achieve connecting Continua AHD to a plurality of remote BT HDP PHD (supporting BT HDP communication protocol standards) through wireless networks having a longer transmission range.

### SUMMARY

The disclosure provides an exemplary embodiment of a distributed application system. According to the exemplary embodiment, the distributed application system includes an application hosting device, a wireless sensor network and a gateway. The application hosting device is connected to a wireless sensor network. The application hosting device includes a personal health manage software module, a first application programming interface module and a first communication protocol module, where the first application programming interface module includes a first portion of functions of a personal area network application programming interface (API). The gateway is connected between the application hosting device and at least one terminal personal health device. The gateway includes a second application programming interface module, a second communication protocol module and a third communication protocol module. The second application programming interface module includes a second portion of functions of the personal area network application programming interface (API). The wireless sensor network, the first and second communication protocol modules both support a first communication protocol. The third communication protocol module supports a second communication protocol. The first and second application programming interface modules collaboratively achieve all functions of the personal area network application programming interface (API), and the all functions is a combination of the first and second portion of functions. Additionally, the at least one terminal personal health device is connected to the application hosting device through the gateway. When the application hosting device or the gateway transmits a message to the wireless sensor network, a transmission quality of service (QoS) control mechanism is performed by setting a transmission priority level and a positive acknowledgement mode of QoS parameters of the message.

The disclosure provides an exemplary embodiment of a method for controlling quality of service in data transmission. According to the exemplary embodiment, the driving method is applicable to a distributed application system comprising an application hosting device, a wireless sensor network and a gateway, and the method includes the following steps. The application hosting device transmits a message to at least one terminal personal health device through the wireless sensor network and the gateway. The application hosting device and the gateway both support a first communication protocol. The at least one terminal personal health device is connected to the application hosting device through the gateway. The at least one terminal personal health device and the gateway both support a second communication protocol. The at least one terminal personal health device transmits another message to the application hosting device through the gateway and the wireless sensor network. Additionally, when the application hosting device or the gateway transmits a message to the wireless sensor network, the application hosting device or the gateway performs a transmission quality of service (QoS) control mechanism by setting a transmission priority level and a positive acknowledgement mode of QoS parameters of the message.

Several exemplary embodiments accompanied with figures are described in detail below to further describe the disclosure in details.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1A is a functional block diagram of a distributed application system according to an embodiment of the disclosure.

FIG. 1B is a detailed functional block diagram of a distributed application system according to an embodiment of the disclosure.

FIG. 2 is a schematic diagram illustrating message fields of a message according to an embodiment of the disclosure.

FIG. 3 is a schematic diagram illustrating packet segmentation according to an embodiment of the disclosure.

FIG. 4 is a schematic diagram illustrating packet reassembly according to an embodiment of the disclosure.

FIG. 5 is a system architecture diagram of a distributed application system according to another embodiment of the disclosure.

FIG. 6 is a flowchart of a method for controlling quality of service in data transmission according to an embodiment of the disclosure.

### DETAILED DESCRIPTION OF DISCLOSED EMBODIMENTS

Below, exemplary embodiments will be described in detail with reference to accompanying drawings so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

QoS (quality of service) control mechanisms are not taken into account in current technology applied to gateways transferring network packets over the wireless sensor network. For example, warning/notification messages and physiological signals transmission/response messages are two different message types so that they need different delay and packet loss QoS requirement when transmitted to wireless sensor network. Thus, the wireless sensor network gateways of current technology performing conversion of different communication protocols is not suitable for transmitting physiological measurement signals or command/warning messages which have requirements on quality of service (QoS).

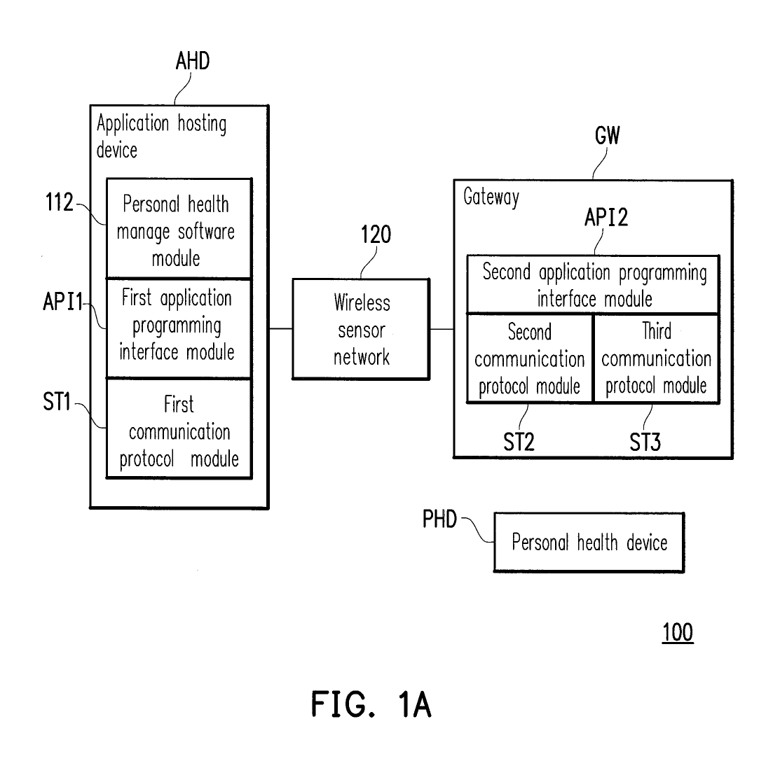

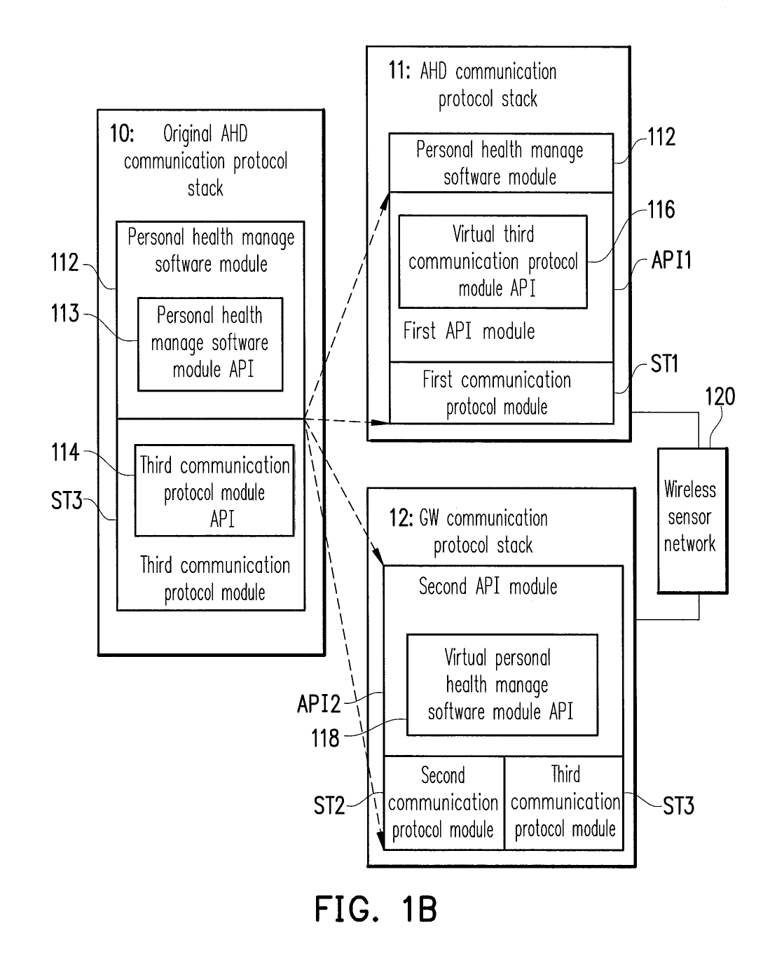

FIG. 1A is a functional block diagram of a distributed application system according to an embodiment of the disclosure. Referring to FIG. 1A, the distributed application system 100 of this embodiment includes an application hosting device AHD, a wireless sensor network 120 and a gateway GW. The application hosting device AHD may be responsible for connecting back-end server system of the distributed application system 100, and meanwhile responsible for collecting front-end physiological measurement data in the distributed application system 100. The application hosting device AHD includes a personal health manage software module 112, a first application programming interface (API) module API1 and a first communication protocol module ST1. The personal health manage software module 112 may includes ISO/IEEE 11073 PHDC (personal health device communication) manager standards which are the application profiles of Continua Compliant AHD. The first API module API1 includes a portion of functionalities of a personal area network API (application programming interface).

The gateway GW includes a second API module API2, a second communication protocol module ST2 and a third communication protocol module ST3. The gateway GW is connected to the application hosting device AHD through the second communication protocol module ST2 and the wireless sensor network 120, and connected to at least one personal health device PHD through the third communication protocol module ST3. The wireless sensor network 120, the first communication protocol module ST1 and the second communication protocol module ST2 all support a first communication protocol. The third communication protocol module ST3 supports a second communication protocol. The first communication protocol may be, for example, ZigBee/802.15.4 communication protocol, but possible implementations of the present disclosure are not limited thereto. The second communication protocol may be a wired communication protocol or a wireless communication protocol.

The first API module API1 allocated in the application hosting device AHD and the second API module API2 allocated in the gateway GW collaboratively achieve all functions of the personal area network API (application programming interface). All functions of the personal area network API includes the all interface functions between the personal health manage software module 112 and the third communication protocol module ST3, in which the personal health manage software module 112 is put on top of the third communication protocol module ST3 directly. The at least one terminal personal health device PHD may be connected to the application hosting device AHD through the gateway GW. In other words, the original interface functions between the personal health manage software module 112 and the third communication protocol module ST3, in which the personal health manage software module 112 is put on top of the third communication protocol module ST3 directly, are replaced by the first API module API1 and the second API module API2 when the personal health manage software module 112 and the third communication protocol module ST3 are separated and settled on the application hosting device AHD and the gateway GW connected by wireless sensor network. Thus, one or more personal health device PHD may transmit command message, physiological measurement message or warning message to the application hosting device AHD through the gateway GW and the wireless sensor network 120. From another perspective, when the first API module API1 transmits at least one interface message of the personal health manage software module 112 to the wireless sensor network 120 through the first communication protocol module ST1 and the second API module API2 transmits at least one interface message of the third communication protocol module ST3 to the wireless sensor network 120 through the second communication protocol module ST2, a transmission quality of service (QoS) control mechanism is provided. For example, the transmission QoS control mechanism is base on the QoS attributes of <transmission priority level/positive acknowledgement (ACK) mode>.

According embodiments of the disclosure, the application hosting device AHD may be a personal computer, a workstation computer, a host computer, or computer in other forms or processors in other forms, but possible implementation of the disclosure is not limited thereto. According embodiments of the disclosure, the application hosting device AHD may be allocated in a hospital, a ward, a house, a nursing home, or a clinic. Also, the application hosting device AHD may be a server managing one or more terminal personal heath devices PHD. The personal heath devices PHD may be electronic medical devices such as a blood glucose meter, a blood pressure meter, a blood lipids meter, a blood oxygen concentration meter, an Electrocardiography (ECG) physiological signal measurement device, or an electronic body temperature meter, but possible implementations of the disclosure are not limited thereto.

According to embodiments of the disclosure, the gateway GW may be a mobile electronic device such as a mobile phone, a tablet computer, a personal digital assistant (PDA), notebook computer, a smart phone, or a handheld smart device. Alternatively, the gateway GW may be a fixed location electronic device such as a personal computer, a workstation computer or computers in other forms, but possible implementations of the disclosure are not limited thereto.

The gateway GW supports the second communication protocol to connect at least one personal health device PHD. The second communication protocol may be, for example, wireless bluetooth health device profile (BT HDP) or wired universal serial bus personal healthcare device class (USB PHDC) communication protocol, but possible implementations of the disclosure are not limited thereto.

According to an embodiment of the disclosure, the application hosting device AHD may use its first communication protocol module ST1 to communicate with the second communication protocol module ST2 in the gateway GW through the wireless sensor network 120. Also, the first API module API1 may receive a first message generated by the personal health manage software module 112 in an upper layer and convert the first message into a second message supporting the first communication protocol. Further, the first API module API1 may transmit the second message to the sensor network 120 by using the first communication protocol module ST1. Additionally, the first communication protocol module ST1 is in a lower layer in relation to the first API module API1 in a communication protocol stack.

The personal health manage software module 112 in an upper layer of the first API module API1 may include: IEEE 11073 PHD protocol stack and Continua AHD application programs on top of the IEEE 11073 PHD protocol stack. The first communication protocol module ST1 is in a lower layer of the first API module API1 in the communication protocol stack. The first communication protocol module ST1 may include: IEEE 802.15.4 physical layer (PHY), IEEE 802.15.4 medium access control (MAC) layer on top of the IEEE 802.15.4 PHY layer, and ZigBee communication protocol stack on top of the IEEE 802.15.4 MAC layer.

Similarly, the gateway GW may include a second API module API2, a second communication protocol module ST2 and the third communication protocol module ST3. The second communication protocol module ST2 may include IEEE 802.15.4 PHY layer, IEEE 802.15.4 MAC layer on top of the IEEE 802.15.4 PHY layer and ZigBee communication protocol stack on top of the IEEE 802.15.4 MAC layer. The gateway GW may use the second communication protocol module ST2 to communicate with the first communication protocol module ST1 of the application hosting device AHD through the wireless sensor network 120. Also, the second API2 module API2 may receive the second message transferred by the second communication protocol module ST2 in a lower layer in relation to the second API module API2 in a communication protocol stack, and convert the second message into a third message supporting the second communication protocol. Further, the second API module API2 transmits the third message to one or more terminal personal health devices (such as the personal health device PHD in FIG. 1A) through a third communication protocol module ST3 in a lower layer in relation to the second API module API2 in a communication protocol stack.

In one embodiment of the disclosure, the gateway GW may use the second communication protocol module ST2 to communicate with the first communication protocol module ST1 in the application hosting device AHD. Also, the gateway GW may use the third communication protocol module ST3 to receive a fourth message transmitted from one of the terminal personal health devices (such as the personal health device PHD in FIG. 1), where all terminal personal health devices support the second communication protocol. The fourth message supports the second communication protocol as well. Further, the second API module API2 of the gateway GW converts the fourth message into a fifth message supporting the first communication protocol, and transmits the fifth message to the first communication protocol module ST1 through the second communication protocol module ST2 and the wireless sensor network 120. Then, the first API module API1 of the application hosting device AHD receives the fifth message through the first communication protocol module ST1, and the first API module API1 converts the fifth message into a sixth message. Additionally, the first API module API1 transfers the sixth message to the personal health manage software module 112, and the personal health manage software module 112 may process the sixth message subsequently.

Further, in other embodiments, the message format supported by the personal health manage software module 112 in the application hosting device AHD may be identical to the message format of the application profile supported by the personal health device PHD. Thus, when the personal health manage software module 112 in the application hosting device AHD intends to communicate with the personal health device PHD, the first API module API1 may firstly converts the first message transmitted by the personal health manage software module 112 into the second message (of the first communication protocol) which may be transmitted by the first communication protocol module ST1. Then, the second message which is in a converted format may be transmitted by the first communication protocol module ST1 to the second communication protocol module ST2 in the gateway GW, and the second communication protocol module ST2 and the second API module API2 may process the second message subsequently. The second message may be further converted by the second API module API2 into a third message (of the second communication protocol) which may be subsequently transmitted by the third communication protocol module ST3. Additionally, the third message may be transmitted to the personal health device PHD through a communication interface between the third communication protocol module ST3 and the personal health device PHD.

On the contrary, when the first API module API1 receives the fifth message in a message format of the first communication protocol from the wireless sensor network 120, the first API module API1 may convert the fifth message into the sixth message in another message format supported by the personal health manage software module 112, and the sixth message in a converted message format may be transmitted up to the personal health manage software module 112 for subsequent message processing.

FIG. 1B is a schematic diagram illustrating a protocol stack of a distributed application system according to an embodiment of the disclosure. The left-hand side (LHS) of FIG. 1B is an original AHD communication protocol stack 10. The personal health manage software module 112 provides a personal health manage software module API 113 for the third communication protocol module ST3 in a lower layer to use, that is, the third communication protocol module ST3 may transmit a message to the personal health manage software module 112 through the personal health manage software module API 113. The third communication protocol module ST3 provides a third communication protocol module API 114 for the personal health manage software module 112 in a top layer to use, that is, the personal health manage software module 112 may transmit a message to the third communication protocol module ST3 through the third communication protocol module API 114. The combination of the personal health manage software module API and the third communication protocol module API forms the personal area network API. The right-hand side (RHS) of FIG. 1B is a distributed application system transformed by an original AHD. In the distributed application system of the RHS, the application hosting device AHD may includes AHD communication protocol stack 11 and the gateway GW includes GW communication protocol stack 12. The first API module API1 of the application hosting device AHD includes a virtual third communication protocol module API 116 which is the first portion of functions of the personal area network API. The API which is the same as the third communication protocol module API 114 is provided for the personal health manage software module 112 on a top layer to use. The second API module API2 of the gateway GW includes a virtual personal health manage software module API 118 which is the second portion of functions of the personal area network API. The API which is the same as the personal health manage software module API 113 is provided for the third communication protocol module ST3 to use. In addition, the first API module API1 and the second API module API2 collaboratively achieve all functions of the personal area network API, and the combination of said first portion functions and said second portion of functions forms all functions of the personal area network API. The above-mentioned first portion functions represents that the first API module API1 provides the virtual third communication protocol module API 116 for the personal health manage software module 112 to use. And the effect of the virtual third communication protocol module API 116 is the same as that of the third communication protocol module API 114. The above-mentioned second portion functions represents that the second API module API2 provides the virtual personal health manage software module API 118 for the third communication protocol module ST3 to use. And the effect of virtual personal health manage software module API 118 is the same as that of the personal health manage software module API 113. Namely, because of the design of the virtual third communication protocol module API 116 and that of the virtual personal health manage software module API 118, one or more terminal personal health devices may transmit the PHD signals, the physiological measurement signals or the command/warning messages to the application hosting device AHD through the gateway GW and the wireless sensor network 120. On the other hand, when the first and second API modules API1 and API2 respectively transmit the messages to the wireless sensor network through the first and second communication protocol modules ST1 and ST2, the first and second API modules API1 and API2 will perform QoS control mechanism.

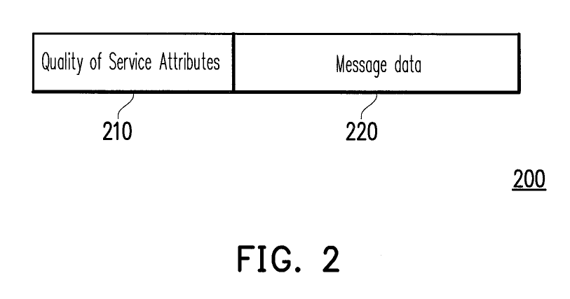

FIG. 2 is a schematic diagram illustrating message fields of a message 200 according to an embodiment of the disclosure. Referring to FIG. 2, in the present embodiment, the message 200 may be in a network packet payload format supporting the first communication protocol. The message 200 may include a QoS attribute field 210 and a message data field 220. The <Reliability, Latency> QoS attribute field may be obtained by analyzing the message data. Meanwhile, according to the <Reliability, Latency> QoS attribute field in Table II, <Transmission priority level/Transmission mode/positive ACK mode> QoS control parameters may be obtained correspondingly, but possible implementation of the disclosure is not limited thereto. Moreover, the QoS attribute field 210 may label QoS attributes of the message 200, and the message data field 220 may store/carry message data of the message 200. The transmission priority level of the message 200 represents a priority order of the message 200 to be transmitted comparing with other messages, and the positive acknowledgement mode of the message 200 indicates whether a communication device receiving the message needs to return a positive acknowledgement packet.

During the process of transforming the first message into the second message by the first API module API1, the first API module API1 classifies messages according to the second field (message type) in Table II, determines the message type according to the content of the message, so as to decide <Reliability, Latency> QoS attributes and corresponding <Transmission priority level/Transmission mode/positive ACK mode> QoS control parameters of transmitting the second message to the wireless sensor network 120, and performs the transmission QoS control mechanism according to the QoS parameters alone or with a QoS control function of the first communication protocol module ST1. The first API module API1 and the first communication protocol module ST1 may collaboratively perform the QoS control function according to the QoS control parameters in the application hosting device AHD.

The transmission mode of the first communication protocol module ST1 includes a contention mode and a guaranteed timeslot mode. When the first API module API1 uses the contention mode for transmitting the second message, the first API module API1 also determines whether to use the positive acknowledgement mode according to the reliability QoS attributes.

The second API module API2 classifies messages according to the second field (message type) in Table II, determines the message type according to the content of the message, so as to decide <Reliability, Latency> QoS attributes and corresponding <Transmission priority level/Transmission mode/positive ACK mode> QoS control parameters of transmitting the fifth message to the wireless sensor network 120, and performs the transmission QoS control mechanism according to the QoS control parameters alone or with a QoS control function of the second communication protocol module ST2. The second API module API2 and the second communication protocol module ST2 may collaboratively achieve the function of controlling the messages to be transmitted which is controlled by the gateway GW according to the QoS parameters.

When the second API module API2 uses the contention mode for transmitting the fifth message, the second API module API2 also determines whether to use the positive acknowledgement mode according to the reliability QoS attribute.

In one embodiment of the disclosure, the first API module API1 simultaneously performs segmentation and re-assembly of packets converted between the first communication protocol and the second communication protocol, but possible implementation of the disclosure is not limited thereto. In another embodiment of the disclosure, the second API module API2 simultaneously performs segmentation and re-assembly of packets converted between the first communication protocol and the second communication protocol.

When the first API module API1 receives the first message from the personal health manage software module 112, the first API module API1 determines the QoS attributes of the message, converts the first message to the second message payload format of the message 200, selects to configure a transmission priority level, a positive acknowledgement mode and a transmission mode of the first communication protocol of transmitting the second message according to the QoS attributes, and performs QoS control mechanism to transmit the second message to the wireless sensor network 120 through the first communication protocol module ST1. After the second API module API2 receives the second message from the second communication protocol module ST2, a priority for subsequent message processing may be decided by the QoS attributes of the second message payload format of the message 200. If the first communication protocol module ST1 does not provide the QoS control function, the first API module API1 may achieve the QoS control function independently. If the first communication protocol module ST1 provides some QoS control function, the first API module API1 may collaborate with the first communication protocol module ST1 to achieve all QoS control functions.

Similarly, when the second API module API2 receives the fourth message from the third communication protocol module ST3, the second API module API2 determines the QoS attributes of the message, converts the fourth message to the fifth message payload format of the message 200, selects to configure a transmission priority level, a positive acknowledgement mode and a transmission mode of the first communication protocol of transmitting the second message according to the QoS attributes, and performs QoS control mechanism through the second communication protocol module ST2 transmitting the fifth message to the wireless sensor network 120. After the first API module API1 receives the fifth message from the first communication protocol module ST1, a priority for subsequent message processing is decided by the QoS attribute of the fifth message payload format of the message 200. If the second communication protocol module ST2 does not provide the QoS control function, then the second API module API2 may achieve the QoS control function independently. If the second communication protocol module ST2 provides some QoS control function, the second API module API2 may collaborate with the second communication protocol module ST2 to achieve all QoS control functions.

The message 200 may be used by the gateway GW to transfer a message generated by the personal health device PHD to the application hosting device AHD. The message generated by the personal health device PHD may be, for example, a measurement report message, a measurement history record message, a physiological alarm message, an equipment alarm message, a measured parameters (such as blood pressure, blood lipid, blood glucose, blood oxygen concentration percentage, heart beat rate, body temperature) message, an event message, a notification message, a request, a response, an equipment control response message (or a status), viewable waveforms (such as ECG waveforms) message and so like.

For example, when the message 200 transmitted from the gateway GW to the application hosting device AHD has QoS attributes of a measured parameters message generated by the personal health device PHD, the receiver may have higher tolerance on transmission delay of such type of measured parameters message. The measured parameters message in this example may carry measured parameters such as blood pressure, blood glucose and blood lipid. The measured parameters message may not need to be transmitted to the receiver in a real-time manner. Therefore, the transmission priority level may be configured at a relatively lower priority level. On the other hand, the accuracy (or reliability) requirement of the measured parameters message should be relatively high. For example, when the personal health device PHD is an electronic body temperature meter, if there is a deviation error between the actual body temperature (for example, 38.5 degrees Celsius) of the user and the measured parameters message collected from the body temperature of the user may be, for example, 37.5 degrees Celsius, the information presented by the measured parameters message at the application hosting device AHD may be wrong (for example, whether the user has a fever). Such wrong information may further mislead medical personnel to arrive at a wrong judgment in physiological status diagnosis. Therefore, the message transmission of the first communication protocol which is configured to transmit the measured parameter message should be high reliability and could be of high latency, but the aforementioned description may serve merely as an example herein. In other embodiment, the configuration of the message transmission may still be correspondingly adjusted according to different implementations or the QoS attributes of the message 200.

In one embodiment of the disclosure, the transmission mode of the first communication protocol may include a contention mode and a guaranteed timeslot mode. The guaranteed timeslot mode in the ZigBee/802.15.4 communication protocol may also be called a contention free period.

The QoS parameters adopted for the first API module API1 of the application hosting device AHD transmitting the second message affects the contending right of accessing the wireless sensor network according to the configured transmission priority level. Similarly, the QoS parameters adopted for the second API module API2 of the gateway GW transmitting the fifth message affects the contending right of accessing the wireless sensor network according to the configured transmission priority level. Messages having different transmission priorities may be respectively stored in different buffers on the first API module API1 of the application hosting device AHD, and transmission priority level for the stored messages in each buffer is identical. Similarly, the second API module API2 of the gateway GW may be allocated different buffers to store messages having different transmission priorities.

For example, if there are allocated three buffers A, B, C in the first API module API1 of the application hosting device AHD (or the second API module API2 of the gateway GW), and the buffers may be configured to respectively store one or more messages respectively having transmission priority levels a, b, c, then the transmission priority order may be configured as following. The transmission priority level a is higher than the transmission priority level b; and the transmission priority level b is higher than the transmission priority level c. For example, all messages configured with the transmission priority level a will be stored in the buffer A; all messages configured with the transmission priority level b will be stored in the buffer B; and all messages configured with the transmission priority level c will be stored in the buffer C.

Thus, if two buffers (for example, the buffers A, C) or more than two buffers among the buffers A, B, C simultaneously have requests of transmitting messages, then the message having a higher transmission priority level (such as the transmission priority level a) will be transmitted according to the priority order; then the messages having a lower transmission priority level (such as the transmission priority level c) will contend for the right of accessing the gateway GW subsequently. That is, the messages having the lower transmission priority level (such as the transmission priority level c) can contend for the right of accessing the gateway GW only after the messages having the higher transmission priority level (such as the transmission priority level a) are all transmitted out.

When the first API module API1 of the application hosting device AHD is configured to transmit the second message, the first API module API1 may determine whether to use the positive acknowledgement mode according to the reliability parameter of the QoS attribute of the second message. When the second API module API2 of the gateway GW is configured to transmit the fifth message, the second API module API2 may determine whether to use the positive acknowledgement mode according to the reliability parameter of the QoS attribute of the fifth message.

For example, if the positive ACK mode is adopted for the message transmission in the contention mode, when the first API module API1 of the application hosting device AHD successfully transmits the message to the second API module API2 of the gateway GW, the gateway GW may reply a positive ACK message to the application hosting device AHD. In this way, the gateway GW notifies the application hosting device AHD that the message is successfully received. When the application hosting device AHD does not receive the positive ACK message from the gateway GW, it means that the message is not successfully delivered to the gateway GW, and the first API module API1 may re-transmit the same message within a predetermined period. On the other hand, when the positive ACK mode is not adopted for message transmission in the contention mode, the gateway GW will not reply the positive ACK message to the application hosting device AHD no matter the message is successfully received by the gateway GW or not.

Similarly, if the gateway GW configures message transmission to adopt the positive ACK mode, the gateway GW likewise may determine whether the transmitted message is successfully received by the application hosting device AHD based on whether receiving the positive ACK message replied by the application hosting device AHD.

The second API module API2 may be configured to receive the second message transferred by the second communication protocol module ST2 in the lower layer of the second API module API2 in the communication protocol stack, and transmit the fifth message to the first communication protocol module ST1 through the second communication protocol module ST2. The fifth message may be, for example, a measurement report message, a measurement history record message, a physiological alarm message, an equipment alarm message, a measured parameters (such as blood pressure, blood lipid, blood glucose, blood oxygen concentration percentage, heart beat rate, body temperature) message, an event message, a notification message, a request, a response, an equipment control response message (or a status), viewable waveforms (such as ECG waveforms) message and so like.

The first API module API1 and the second API module API2 may respectively perform format conversions of network packets of the first communication protocol and the second communication protocol. However, since effective payloads of network packets of the first communication protocol and the second communication protocol are different, the first API module API1 or the second API module API2 is required to perform segmentation or re-assembly on the network packets requiring conversion. Thus, the converted network packet can be compatible with the effective payload of corresponding communication protocol. If the first communication protocol module ST1 provides the function of segmentation and re-assembly of network packets, the first API module API1 achieves segmentation and re-assembly of network packets by using the first communication protocol module ST1. If the second communication protocol module ST2 provides the function of segmentation and re-assembly of network packets, the second API module API2 achieves segmentation and re-assembly of network packets by using the second communication protocol module ST2.

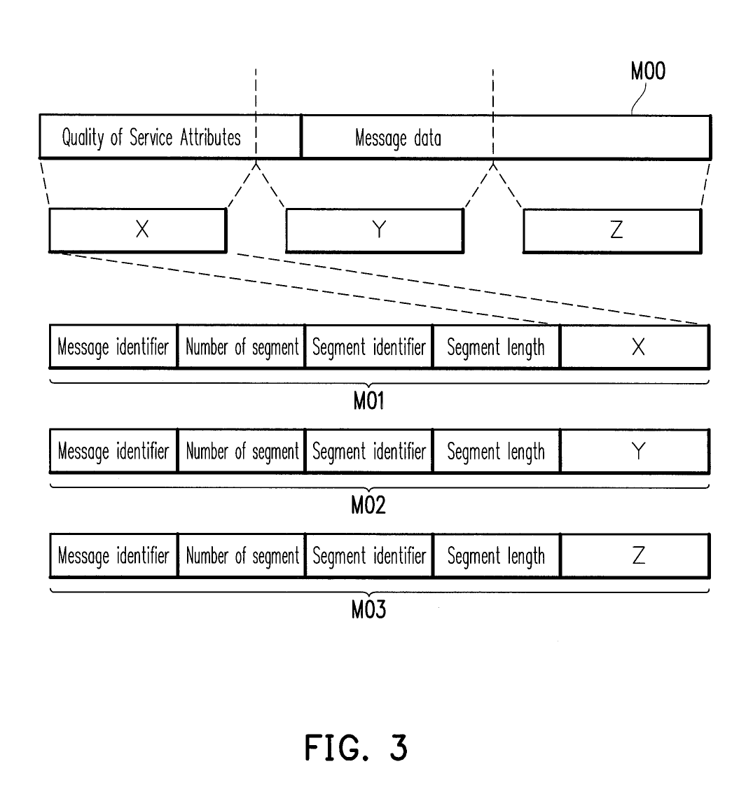

FIG. 3 is a schematic diagram illustrating packet segmentation according to an embodiment of the disclosure. Referring to FIG. 3, in the present embodiment, the network packet payload M00 may be in a network packet payload format of the message 200. If the length of the network packet payload of the message 200, which is transmitted by the first API module API1 or the second API module API2 through the first communication protocol module or the second communication protocol module, is greater than the length (e.g., 96 bytes) of the effective network packet payload of the application layer supported by the first communication protocol, then when the first API module API1 or the second API module API2 converts the network packet payload of the message 200 into the network packet payload supported by the first communication protocol, the network packet payload of the message 200 has to be divided to be compatible with the effective payload of the network packet of the first communication protocol. As shown in FIG. 3, if the length of the network packet payload of the message 200 may be, for example, 200 bytes; the length of the effective payload of the first communication protocol (i.e. Zigbee communication protocol standard) is 96 bytes.

For example when the first API module API1 or the second API module API2 converts the network packet payload M00 into network packet(s) payload of the first communication protocol, the original network packet payload M00 may be divided into three sub-packets payload X, Y and Z, relevant information regarding network packet re-assembly may be added during a packet segmentation process, such that the network packets generated from the packet segmentation process may be accurately re-assembled at the receiver side, so as to recover original information of the packet content. It is further illustrated with a simplified example below.

Referring again to FIG. 3, if the length of the network packet payload of the message 200 is, for example, 256 bytes, after a packet identifier (Message_ID) field, a number of segment (Num_Of_Segment) field, a segment identifier (Segment_ID) field, and a segment length (Segment_Len) field associated with network packet segmentation and re-assembly are being added to the sub-packet payloads X, Y and Z respectively, network packets payloads M01, M02, M03 supporting the first communication protocol are respectively formed. The packet identifier (Message_ID) field, the number of segment (Num_Of_Segment) field, the Segment identifier (Segment_ID) field, and the Segment length (Segment_Len) field of the network packet payloads M01, M02, M03 may be presented according to configuration shown in Table I below. When the length of the four fields are assumed to be 1 bytes and the length of the sub-packet payload is assumed to be 92 bytes, the length of the resultant network packet payload supporting the first communication protocol may be a maximum of 1×4+92=96 bytes. In other embodiments, the length and configuration of contents in these fields may be presented in different formats, and other information field(s) may also be added to more specifically record information of segmentation and re-assembly of the network packet payload.

TABLE I

| | Packet | Packet | Packet |
|---|---|---|---|
| | payload M01 | payload M02 | payload M03 |
| Length of Message_ID | 1/15 | 1/15 | 1/15 |
| field/value | | | |
| Length of | 1/3 | 1/3 | 1/3 |
| Num_Of_Segment | | | |
| field/value | | | |
| Length of Segment_ID | 1/1 | 1/2 | 1/3 |
| field/value | | | |
| Length of | 1/92 | 1/92 | 1/72 |
| Segment_Len field/ | | | |
| value | | | |
| Length of sub-packet | 92 | 92 | 72 |
| payload | | | |
| Total length of | 96 | 96 | 76 |
| network packet | | | |
| payload | | | |
| Effective payload | 96 | 96 | 76 |
| length of Zigbee | | | |
| application layer | | | |

Referring to both FIG. 3 and Table I, when the message identifier of the network packet payload M00 configured by the first API module API1 or the second API module API2 is 15, contents of the message identifier in the network packet payload M01, M02, M03 are all configured to be 15. Here, the message identifier is configured to indicate that the sub-packet payloads X, Y and Z respectively in the network packet payload M01, M02, M03 generated by segmentation of the network packet payload M00 which has a network packet identifier of “15”. The number of segment (Num_Of_Segment) field in Table I may be configured equivalently according to the number of segments generated by segmentation of the network packet payload M00. In the present embodiment, since the network packet payload M00 is assumed to be segmented into three sub-packet payloads X, Y and Z, the content of the number of segment (Num_Of_Segment) field in the network packet payload M01, M02, M03 may be correspondingly configured to be 3 (which may refer to three segments). The segment identifier (Segment_ID) field in Table I may be configured according to an order of the sub-packet payloads X, Y and Z of the network packet payload M01, M02, M03 in the network packet payload M00. The order may be, for example, an order of the content from the beginning of the network packet payload to the end of the network packet payload, and may be even labeled to indicate in what kind of sequence the sub-packets X, Y and Z should be re-assembled. The content of the segment length (Segment_Len) field in Table I may be configured according to packet lengths of the sub-packet payloads X, Y and Z in the network packet payload M01, M02, M03. Since packet lengths of the sub-packet payloads X, Y and Z in the present embodiment are configured to be 92 bytes, 92 bytes and 72 bytes, the content of the Segment length (Segment_Len) field of the network packet payload M01, M02, M03 can be correspondingly configured to be 92 bytes, 92 bytes and 72 bytes.

In one embodiment of the disclosure, the first API module API1 may simultaneously perform segmentation of the network packets when transmitting the second message to the wireless sensor network and perform re-assembly of network packets when receiving the fifth message from the wireless sensor network. The second API module API2 may simultaneously perform segmentation of the network packets when transmitting the fifth message to the wireless sensor network and perform re-assembly of network packets when receiving the second message from the wireless sensor network. If the first communication protocol module ST1 and the second communication protocol module ST2 provide the segmentation and re-assembly functions, then the first API module API1 and the second API module API2 do not need to provide the segmentation and re-assembly functions and may directly use the segmentation and re-assembly functions provided by the first and second communication protocol modules ST1 and ST2.

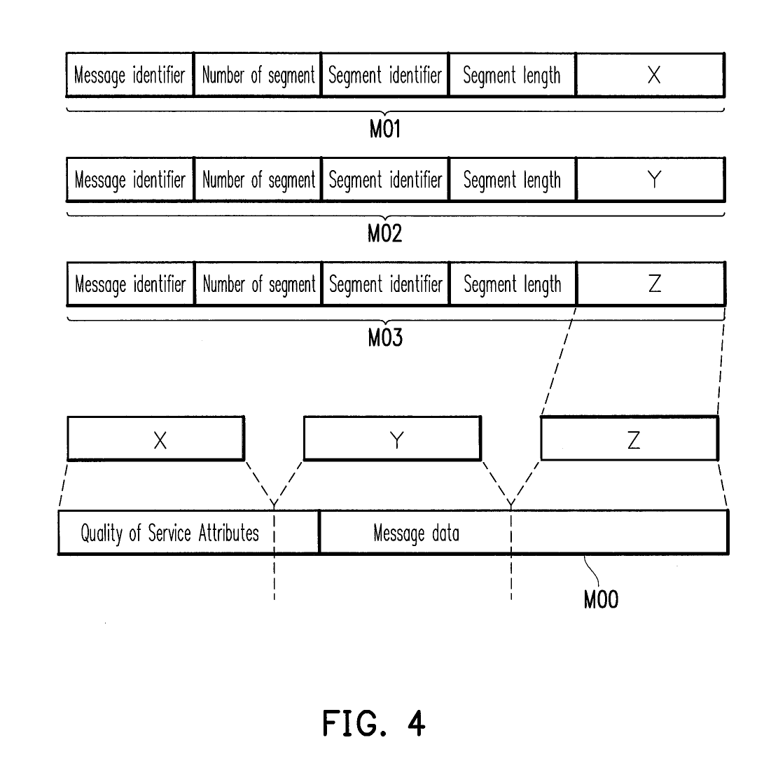

FIG. 4 is a schematic diagram illustrating packet reassembly according to an embodiment of the disclosure. Referring to FIG. 4, it is assumed that the content of the packet identifier (Message_ID) field, the number of segment (Num_Of_Segment) field, the Segment identifier (Segment_ID) field, and the Segment length (Segment_Len) field of the network packet payload M01, M02, M03 may be respectively configured according to the content in Table I. Then, after receiving the network packet payload M01, M02, M03, the first API module API1 or the second API module API may retrieve the sub-packet payloads X, Y and Z in the network packet payload M01, M02, M03 and re-assemble the sub-packet payloads X, Y and Z into a packet payload M00 based upon information respectively provided by fields of the network packet payload M01, M02, M03 according to Table I.

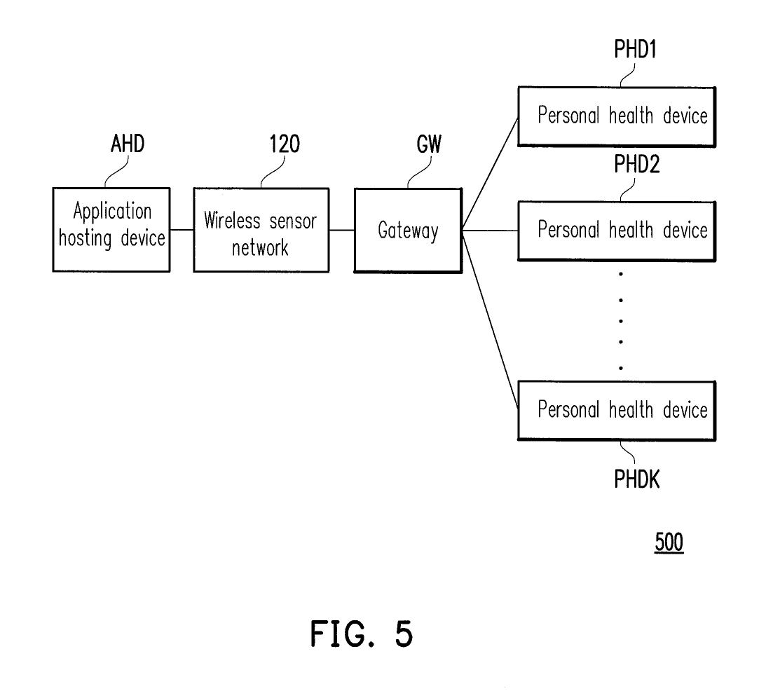

FIG. 5 is a functional block diagram of a distributed application system according to another embodiment of the disclosure. Referring to FIG. 5, in a distributed application system 500, a gateway GW may be connected through a wired communication interface or a wireless communication interface with a plurality of personal health devices PHD1˜PHDK, where K is a positive integer greater than 1. The wired communication interface is, for example, USB PHDC interface; and the wireless communication interface is, for example, BT HDP interface.

It is assumed that the application hosting device AHD (e.g., a server which is configured to centrally manage information of the personal health devices PHD1˜PHDK) is disposed in a living room of a medical building. Also, the personal health devices PHD1˜PHDK (e.g., blood pressure meter, blood glucose meter or so like) are assumed to be disposed in other rooms of the same medical building. Further, the gateway GW may be disposed in other rooms of the same medical building, in the living room and the aisles. Besides, the application hosting device AHD and the personal health devices PHD1˜PHDK may be in different level other than the level where the application hosting device AHD is disposed.

When the first communication protocol is ZigBee/802.15.4 communication protocol, and the second communication protocol is Bluetooth health device profile (BT HDP) communication protocol, the personal health devices PHD1˜PHDK supporting the Bluetooth communication protocol and disposed at home is intended to communicate with application hosting device AHD in a longer distance, the personal health devices PHD1˜PHDK may firstly communicate with the gateway GW through the Bluetooth protocol, and then communicate with the application hosting device AHD at remote site through the gateway GW by ZigBee/802.15.4 communication protocol. Here, the longer distance may refer to a distance less than or equal to the communication range of the ZigBee network but greater than the transmission range of the Bluetooth. Accordingly, the personal health devices PHD1˜PHDK may obtain a longer transmission range through the ZigBee communication protocol supported by the gateway GW. That is, through conversion of the gateway GW, the personal health devices PHD1˜PHDK merely supporting the Bluetooth communication protocol may transmit messages to the application hosting device AHD at remote site through the network supporting the ZigBee/802.15.4 communication protocol and the gateway GW. On the other hand, the application hosting device AHD may also transmit set message, get message, request message, response message or control action message to the personal health devices PHD1˜PHDK merely supporting the Bluetooth communication protocol through the wireless sensor network supporting the ZigBee/802.15.4 communication protocol and the gateway GW.

TABLE II

| | | QoS control parameters |
|---|---|---|
| QoS attributes | | <Transmission priority |
| <Reliability. | | level/Transmission |
| Latency> | Message Type | mode/Positive ACK mode> |
| <Best. Very High> | Measurement report, | <Low/Contention |
| | Measurement history | mode/APS_ACK=YES> |
| | record | |
| <Best. High> | Physiological driver | <Medium/Contention |
| | alarm, equipment | mode/APS_ACK=YES> |
| | issued alarm | |
| <Best. Medium> | Set command, Get | <High/Contention |
| | command, Event | mode/APS_ACK=YES> |
| | message, Notification | |
| | message, Request, | |
| | Response, Control | |
| | action message, | |
| | Status message | |
| <Better. Medium> | Measured parameters | <High/Contention |
| | (e.g., blood pressure, | mode/APS_ACK=YES> |
| | blood oxygen | |
| | concentration rate, | |
| | heart beat rate or | |
| | so like) | |
| <Good. Medium> | Viewable | <VeryHigh/Contention |
| | waveform(e.g., ECG | mode/APS_ACK=NO> |
| | streaming) | |
| <Good. Low> | | <Highest/Contention |
| | | mode/APS_ACK=NO> |

The six combined values of QoS attributes and message type in the first and second fields of Table II is formulated by Continua Guideline. Because Continua Guideline does not formulate the <Good. Low> QoS attribute now, the corresponding message type in Table II has not been defined, and thus five message types are provided in Table II. In the present embodiment, the designed combined value of QoS parameters (e.g., transmission priority level/transmission mode/positive ACK mode) in the third field of Table II are designed corresponding to the combined value of the first field of Table II. The first API module API1 of the application hosting device AHD and the second API module API2 of the gateway GW may transmit messages to the personal health devices PHD1˜PHDK through the non-data/control connections or data connections of the third communication protocol module ST3. The third communication protocol module ST3 is a transport layer communication module, and thus the message of the non-data/control connection is a related command message (e.g., the related command message of BT HDP and USB PHDC) of the transport layer. The combined value of QoS attributes of the non-data/control connection message may be configured in default value and classified into the message type of <Best. Medium> QoS attributes. The message of the data connection is a related personal health manage software module message, the message content includes data connection identifier and application profile layer message payload (e.g., IEEE 11073 PHD message payload), and the message may be classified into five message types in Table II by analyzing message content. When it is determined that the second message or the fifth message is the non-data connection message, the combined value of QoS control parameters of the message may be configured in default value. When it is determined that the second message or the fifth message is the data connection message, the combined value of QoS control parameters may be configured by analyzing connection identifier and other payloads and by adopting QoS mapping table. In the present embodiment, the reliability of the message may be classified into three types (Best, Better, Good); the latency of the message may be classified into four types (Very High, High, Medium, Low).

Although the three reliability levels and four latency levels may combined to form twelve combined values of QoS attributes, there are six combined values of QoS attributes have been adopted according to the content of Continua Guideline. The combined value of <Reliability, Latency> QoS attributes may be <Best, Very high>, <Best high>, <Best, medium>, <Better, medium>, <Good, medium> and <Good, low>.

The relationship between the <Reliability, Latency> QoS attributes and the <Transmission priority level/Transmission mode/Positive ACK mode> QoS parameters is illustrated in the first field and the third field in Table II. The latency QoS attribute is corresponding to transmission priority level QoS parameter mainly, and the reliability QoS attribute is corresponding to transmission mode and positive ACK mode parameters. Since most of the wireless sensor network do not support non-contention mode, the contention mode is selected in Table II and the best reliability QoS attribute is corresponding to positive ACK mode. The QoS attribute for transmitting blood pressure, blood sugar, blood oxygen etc. measured parameters in Table II is <Better. Medium>. However, the transmission loss of the measured parameters (e.g., blood pressure, blood sugar, blood oxygen etc.) is not desired to happen in practice, and thus the reliability QoS attributes is essentially upgraded to the best and the QoS control parameters are set to adopt positive ACK mode.

Regarding messages of the six combined values of QoS attributes, in the present embodiment, the six combined values of <Transmission priority level/Transmission mode/Positive ACK mode> QoS control parameters may be configured as <Low/Contention mode/APSACK=YES>, <Medium/Contention mode/APS_ACK=YES>, <High/Contention mode/APS_ACK=YES>, <High/Contention mode/APS_ACK=YES>, VeryHigh/Contention mode/APS_ACK=NO>, <Highest/Contention mode/APS_ACK=NO>. There may be five configured transmission priority levels such as (from low to high): low, medium, high, very high and highest. The messages of five transmission priority levels may be respectively stored in five different buffers on the first API module API1 of the application hosting device AHD (or the second API module API2 of the gateway GW). These buffers have priority levels in transmitting data in the contention mode (for high to low) such as: highest, very high, high, medium and low. Various types of contention messages may be mapped to corresponding combined value of QoS control parameters according to their QoS attributes.

The second message and the fifth message to be transmitted to the wireless sensor network may be classified into non-data connection message or data connection message. Non-data connection message is a related transport layer (e.g., BT HDP layer) command message, and may be configured in default message type, the combined value of QoS attributes thereof is <Best. Medium>. In the non-data connection message, all the information of the data connection (e.g., data connection identifier, connection establishing status, QoS attributes or so like) can be monitored, and established data connection may be recorded in QoS mapping table according to the information. Data connection message may includes the application profile layer (e.g., IEEE 11073 PHD layer) data payload, and the message content includes data connection identifier and application profile layer data payload and may be classified into various message types in Table II.

If the second communication protocol is USB PHDC communication protocol, this means that the third communication protocol module ST3 supports USB PHDC communication protocol. The first API module API1 may observe the related USB pipe information and determine whether a new data connection (USB pipe connection) is established or not, and establish the corresponding pipe connection QoS record in QoS mapping table. The six pipe connection QoS attributes are corresponding to the six QoS attributes defined in Continua Guideline directly. The corresponding relationship may be referred to Table III below. For example, the [very high, best] is corresponding to <best, very high>; the [high, best] is corresponding to <best, high>; and the mapping relationship between the rest of [latency, reliability] attribute pairs and the <reliability, latency> QoS attribute pair may be referred to Table III. Since the six pipe connection QoS attributes are corresponding to the six QoS attributes defined in Continua Guideline directly, after the pipe connection is established, if there exists any message to be transmitted in the data connection, the method of controlling QoS parameters of the data connection message may be configured by analyzing data connection identifier and by checking QoS mapping table.

TABLE III

| QoS attribute of USB PHDC pipes | Continua QoS attribute |
|---|---|
| [latency, reliability] | <Reliability, Latency> |
| [Very high, Best] | <Best, Very high> |
| [High, Best] | <Best, high> |
| [Medium, Best] | <Best, Medium> |
| [Medium, Better] | <Better, Medium> |
| [Medium, Best] | <Good, Medium> |
| [Low, Good] | <Good, Low> |

Further, if the second communication protocol is BT HDP communication protocol, this means that the third communication protocol module ST3 supports BT HDP communication protocol. The first API module API1 may observe the establishing status of Bluetooth MDLs (Multi-Channel Adaption Protocol data link) connections according to the content of the first, second, and fifth messages, so as to determine whether a new BT MDL connection is established or not, and then the established bluetooth MDL connection information will be recorded in the QoS mapping table. Since the message in the BT HDP specification are merely classified to be reliable message and streaming message, and the message can not correspond to the six QoS attributes defined in Continua Guideline directly. Except for the MDL identifier information, the message type is determined by also analyzing the data connection message payload (i.e. content of IEEE 11073 PHD message), such that the corresponding QoS control parameters are configured according to the message type in Table II. Another simple method for determining the QoS control parameters is that although the reliable MDL data connection may be corresponding to four combined value of QoS attributes such as <Best, Very high>, <Best high>, <Best, medium> and <Better, medium>, the <Best, medium> QoS attribute is selected merely; although the streaming MDL data connection may be corresponding to two combined values of QoS attributes such as <Good, medium> and <Good, low>, the <Good, medium> QoS attribute is selected merely. The mapping relationship may be referred to Table IV below.

TABLE IV

| BT HDP message | Six Continua QoS attribute |
|---|---|
| mapping rule | <Reliability, Latency> |
| | <Best, Very high> |
| | <Best, high> |
| MDL attribute is Reliable | <Best, Medium> |
| | <Better, Medium> |
| MDL attribute is Streaming | <Good, Medium> |
| | <Good, Low> |

In the present embodiment, the first API module API1 determines that the message is the non-data connection message or the data connection message according to the content of the second message, and adds QoS attribute into the second message during the process of converting the first message into the second message. The first API module API1 may use the default value to configure the QoS attributes and QoS control parameters of the second message of the non-data connection. For the second message of the data connection, the data connection QoS record in the QoS mapping table may be checked to configure the QoS attributes and QoS control parameters. The first API module API1 determines whether a new data connection is established or deleted by observing the content of the fifth, first and second messages, and then establishes or deletes a data connection record in the QoS mapping table. For the second message to be transmitted and belonging to the data connection, the corresponding QoS transmission control may be performed according to the data connection QoS record in the QoS mapping table of the first API module API1.

For example, when a new personal health device PHD (not shown) establishes a new data connection with the gateway GW (e.g., adding a data connection of blood pressure), the gateway GW may transmit the fifth message (e.g., the establishing and enabling messages of the data connection) to the first API module API1 of the application hosting device AHD by using the second communication protocol module ST2, and notify the first API module API1 that a new data connection between the personal health devices PHD and the gateway GW has been established. When the first API module API1 determines that the fifth message is to notify the application hosting device AHD of establishing a new data connection successfully, the first API module API1 adds a new data connection QoS record of the personal health device in the QoS mapping table and configures the transmission priority level, transmission mode and positive ACK mode of the data connection according the QoS attributes of the new data connection.

For example, if the personal health devices PHD transmits a message to the gateway GW through the established data connection, the second API module API2 of the gateway GW may check the QoS mapping table according to the data connection identifier so as to the QoS control parameters of the data connection.

The QoS mapping table may also be presented in a form shown in Table V below. Taking the first API module API1 for example, Table V illustrates that the first API module API1 continuously observes the first, second and fifth messages to obtain the established/deleted status of the data connections, and records the result in the QoS mapping table. Three data connection information with QoS control parameters will be established in the QoS mapping table.

For example, when a simple QoS controlling method is applied to the embodiment rather than conducting a further analysis of IEEE 11073 PHD data payload for BT HDP data connections, a reliable BT HDP data connection is just simply mapping to <Better, Medium> QoS Attributes and a streaming data connection is just simply mapping to <Good, Medium> QoS Attributes, so the messages of the first Bluetooth data connection in Table V whose identifier is #1_MDL_ID will be transmitted according to the corresponding <High transmission priority level/Contention mode/Positive ACK mode> QoS control parameters.

For example, in Table V, the second data connection is a BT HDP streaming data connection, the connection identifier is #2_MDL_ID, and the corresponding QoS parameter is <VeryHigh transmission priority level/Contention mode/Non-positive ACK mode>. It can be deduced that the original BT HDP data connection is a streaming data connection according to the QoS controlling parameter.

For example, in Table V, the third data connection is a BT HDP reliable data connection, the connection identifier is #3_MDL_ID, and the corresponding QoS parameter is high transmission priority level, contention mode, and applying positive ACK mode.

Please referring to FIG. 5 and Table V simultaneously, when the first API module API1 determines that the fifth message received by the first communication protocol module ST1 is the response message which notifies that data connection #1_MDL_ID has been successfully established, the first API module API1 establishes the corresponding connection record of the data connection in the QoS mapping table.

In other embodiments, the first API module API1 may perform the above-mentioned procedure through judging the content of the first or second message. For example, when the data connection (#1_MDL_ID) between the gateway GW and the personal health device has been successfully established, the gateway GW may utilize the second communication protocol module ST2 to transmit the fifth message (for example, notification message) to the first communication protocol module ST1 of the application hosting device AHD to notify the first API module API1. While the first API module API1 determines that the fifth message notifies that the data connection has been successfully established, the first API module API1 establishes and enables the connection record corresponding to the data connection (#1_MDL_ID) in the QoS mapping table (Table V).

TABLE V

| | QoS | | | |
|---|---|---|---|---|
| data | Attributes | Trans- | Contention/ | |
| connection | <Reliability, | mission | Guaranteed | Positive |
| identifier | Latency> | priority | time slot mode | ACK mode |
| #1_MDL_ID | <Better, | High | Contention | ACK mode |
| | Medium> | | mode | adopted |
| #2_MDL_ID | <Good, | VeryHigh | Contention | ACK mode |
| | Medium> | | mode | not adopted |
| #3_MDL_ID | <Better, | High | Contention | ACK mode |
| | Medium> | | mode | adopted |
| . . . | . . . | . . . | . . . | . . . |

In other embodiments, the content of each field in Table V may be presented and configured differently according to application scope of the distributed application system 500.

When the first API module API1 of the application hosting device AHD determines that it is required to transmit a non-data connection message (non IEEE 11073 PHD message) to the wireless sensor network 120, the first API module API1 may directly configure the transmission priority level, the contention/non-contention mode and the positive ACK mode of the control message with default values and without checking the QoS mapping table. That is, when the first API module API1 transmits the non-data connection message through the first communication protocol module ST1, the non-data connection message is configured with fixed and preset configurations such as high transmission priority level, contention mode and adopting the positive ACK mode. However, possible implementation of the present disclosure is not limited thereto. Thus, checking the QoS mapping table is not required but the transmission priority level, the contention/non-contention mode and the positive ACK mode can be directly set according to the preset configuration when transmitting the control action message.

In an embodiment of the disclosure, the second API module API2 of the gateway GW may also include a QoS mapping table. Also, the QoS mapping table of the gateway GW may include identical content (e.g., as shown in Table V) to that of a QoS mapping table of the first API module API1. The second API module API2 determines the message is an non-data connection message (e.g., BT HDP control/command message and the response message thereto) or a data connection message (e.g., IEEE 11073 PHD message) according to the content of the fifth message, and adds QoS attributes into the fifth message during converting the fourth message into the fifth message. The second API module API2 may use the default value to configure the QoS control parameters of the non-data connection message. With regard to the processing of the QoS control parameters of the data connection, it may be performed by the data connection QoS record in the QoS mapping table. The second API module API2 determines whether a new data connection is established and determines the QoS attributes of the data connection according to the content of the fourth, fifth and second messages, and establishes a data connection record in the QoS mapping table. When the second API module API2 determines that it is required to transmit a data connection message to the wireless sensor network 120, the second API module API2 may firstly check the QoS record of the corresponding data connection of this message in the QoS mapping table. Then, the second API module API2 may configure the transmission priority level, the contention/non-contention mode and the positive ACK mode for transmitting this message to the first API module API1.

For example, when the second API module API2 receives a fourth message from the personal health device PHD through the third communication protocol module ST3 is a transport layer (e.g., BT HDP or USB PHDC layer) command response report, the second API module API2 thus determines that it is required to transmit an non-data connection message to the first API module API1, the second API module API2 may use the default value to configure the transmission priority level, the contention/non-contention mode and the positive ACK mode of the non-data connection message without checking the QoS mapping table.

Additionally, when one of the personal health devices PHD1˜PHDK intends to transmit the measured physiological data message (such as blood pressure, blood glucose or body temperature data) to the application hosting device AHD through the gateway GW, the second API module API2 may also check its QoS mapping table when processing the measured physiological data message. To be illustrated more clearly, if the personal health devices PHD1 obtains a physiological data message (such as blood pressure) and transmit such physiological data message to the application hosting device AHD through the established data connection, before the second API module API2 transfers the physiological data message to the application hosting device AHD, the second API module API2 may check the QoS record of the data connection corresponding to the physiological data message. Then, the second API module API2 may decide to set the transmission configuration (e.g., configuration of the transmission priority, whether adopting the contention mode or the positive ACK mode) on the physiological data message, and finally transmit the physiological data message with the decided transmission configuration.

To be illustrated more clearly, if the second API module API2 determines that the forth message received from a personal health device PHD through the third communication module ST3 is to notify that the data connection is successfully established, the second API module API2 may establish a connection record in its QoS mapping table. In other embodiments, the second API module API2 can perform the above-mentioned procedure through judging the content of the second or fifth messages. The second API module API2 may use the default value to configure the QoS attributes and the corresponding transmission priority level, the contention/non-contention mode and the positive ACK mode, convert the fourth message into the fifth message, and transmit the fifth message through the second communication protocol module ST2 and the wireless sensor network 120.

When the second API module API2 determines that the fourth message received through the third communication protocol module ST3 is to notify that the data connection has been deleted, the second API module API2 may delete corresponding connection record of the data connection in the QoS mapping table. In other embodiments, the second API module API2 can perform the above-mentioned procedure through judging the content of the second or fifth messages. Then, the second API module API2 converts the fourth message into the fifth message, and transmits the fifth message to the application hosting device AHD through the second communication protocol module ST2 and the wireless communication network 120.

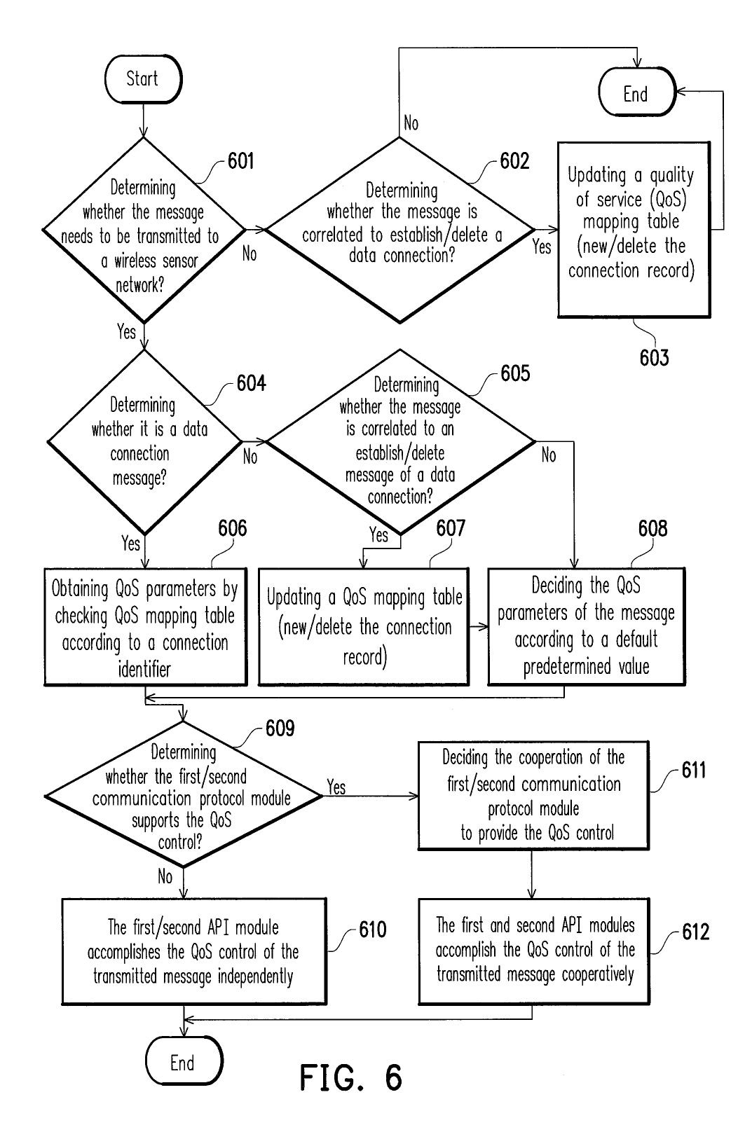

FIG. 6 is a flowchart of a method for controlling quality of service in data transmission according to another embodiment of the disclosure. The method of controlling QoS of the transmission message may adapt to the first API module API1 and the second API module API2.

Below is the description of QoS control flowchart of the data transmission which is processed by the first API module API1. Please referring to FIGS. 1A and 6, in step 601, the first API module API1 of the application hosting device AHD may receive the first message (the message transmitted in the inner modules of the devices is composed of the called function and parameters thereto) from the personal health manage software module 112 in top layer, the fifth message from the first communication protocol module ST1, or a new second message from inside (the second message may be produced or converted in order to response the first/fifth messages). The first and fifth messages does not require transmitting to the wireless sensor network 120, and it will be processed in following steps 602 and 603, but the second message requires transmitting to the wireless sensor network 120. Therefore, when it is determined that the message requires transmitting to the wireless sensor network, step 604 is performed after step 601.

In steps 602 and 603, it is further determined whether the first and fifth messages are correlated to an establishing/deleting a data connection. If yes, the QoS record of the data connection in QoS mapping table is established/deleted according to the content of the message. If no, the method for controlling QoS in data transmission is end here.

In step 604, it is further determined whether the second message belongs to a data connection message (e.g., IEEE 11073 PHD message from personal health manage module 112) or a non-data connection message (e.g., BT HDP or USB PHDC transport layer command or response message thereof). In step 605, since the previous step 604 has confirmed the message is the non-data connection message, it is further determined whether the message includes the related message of establishing/deleting data connection. In step 607, the QoS record of the data connection in QoS mapping table is established/deleted according to the content of the message.

In step 608, since the previous step 604 has confirmed the message is the non-data connection message, the first communication protocol module ST1 of the application hosting device AHD may use the default value to configure the transmission QoS control parameters without checking the QoS mapping table. In step 606, since the previous step 604 has confirmed the message is the data connection message, the transmission QoS parameters of the message are configured by checking the QoS mapping table.

In step 609, since the previous step 601 has confirmed the message requires transmitting to the wireless sensor network 120, the first API module API1 of the application hosting device AHD performs different level of QoS controlling function according to the QoS control functions provide by the first communication protocol module ST1. In step 610, since the previous step 609 has confirmed the first communication protocol module ST1 does not provide the transmission QoS function, the first API module API1 may use the combination of different buffers and positive ACK mode to achieve all the transmission QoS control of <Transmission priority level/Contention or non-contention mode/positive ACK mode>.

In step 611, since the previous step 609 has confirmed the first communication protocol module ST1 provides the transmission QoS function, for example, some of the enhanced 802.15.4 MAC modules use the reserved three bits in MAC header specified in the 802.15.4 MAC specification to provide eight different transmission services, and various re-transmission number parameters may be configured by using the eight different transmission services respectively. Besides, application support sublayer acknowledgement (APS ACK) is provided after Zigbee 2007 version. Therefore, the first API module API1 may decide how to collaborate with the transmission QoS function provided by the first communication protocol module ST1 so as to obtain effective control method. In step 612, the first API module API1 will collaborate with the transmission QoS function provided by the first communication protocol module ST1 to achieve all the transmission QoS control functions of the second message. In step 612 or after step 612, the method for controlling QoS in data transmission is end here.

Below is the description of QoS control flowchart of the data transmission which is processed by the second API module API2. Please referring to FIGS. 1A and 6, in step 601, the second API module API2 of the gateway GW may receive the second message from the second communication protocol module ST2, the fourth message from the third communication protocol module ST3, or a new fifth message produced from inside (the fifth message may be produced or converted in order to response the second/fourth messages). The second and fourth messages does not require transmitting to the wireless sensor network 120, and it will be processed in following steps 602 and 603, but the fifth message requires transmitting to the wireless sensor network 120. Therefore, when it is determined that the message requires transmitting to the wireless sensor network, step 604 is performed after step 601.

In steps 602 and 603, it is further determined whether the second and fourth messages are correlated to an establishing/deleting message of a data connection. If yes, the QoS record of the data connection in QoS mapping table is established/deleted according to the content of the message. If no, the method for controlling QoS in data transmission is end here.

In step 604, it is further determined whether the fifth message belongs to a data connection message (e.g., IEEE 11073 PHD message to personal health manage module 112 of Application Hosting Device AHD) or a non-data connection message (e.g., BT HDP or USB PHDC transport layer command or response message thereof). In step 605, since the previous step 604 has confirmed the message is the non-data connection message, it is further determined whether the message includes the related message of establishing/enabling/disabling/disconnecting data connection.

In step 607, the QoS record of the data connection in QoS mapping table is established/deleted according to the content of the message. In step 608, since the previous step 604 has confirmed the message is the non-data connection message, the second API module API2 of the gateway GW may use the default value to configure the transmission QoS parameters without checking the QoS mapping table. In step 606, since the previous step 604 has confirmed the message is a data connection message, the transmission QoS parameters of the message are configured by checking the QoS mapping table.

In step 609, since the previous step 601 has confirmed the message requires transmitting to the wireless sensor network 120, the second API module API2 perform different level of QoS controlling function according to the QoS control functions provide by the second communication protocol module ST2. In step 610, since the previous step 609 has confirmed the second communication protocol module ST2 does not provide the transmission QoS function, the second API module API2 may use the combination of different buffers and positive ACK mode to achieve all the transmission QoS control of <Transmission priority level/Contention or non-contention mode/Positive ACK mode>.

In step 611, since the previous step 609 has confirmed the second communication protocol module ST2 provides the transmission QoS function, for example, some of the enhanced 802.15.4 MAC modules use the reserved three bits in MAC header specified in the 802.15.4 MAC specification to provide eight different transmission services, and various re-transmission number parameters may be configured by using the eight different transmission services respectively. Besides, application support sublayer acknowledgement (APS ACK) is provided after Zigbee 2007 version. Therefore, the second API module API2 may decide how to collaborate with the transmission QoS function provided by the second communication protocol module ST2 so as to obtain effective control method. In step 612, the second API module API2 will collaborate with the transmission QoS function provided by the second communication protocol module ST2 to achieve all the transmission QoS control functions of the second message.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. A distributed application system, comprising:
an application hosting device, connected to a wireless sensor network, comprising a personal health manage software module, a first application programming interface module and a first protocol communication module, wherein the first application programming interface module comprises a first portion of functions of a personal area network application programming interface (API);
a wireless sensor network; and
a gateway, connected to a wireless sensor network and at least one terminal personal health device, comprising a second application programming interface module, a second communication protocol module and a third communication protocol module,
wherein the second application programming interface module comprises a second portion of functions of the personal area network application programming interface (API), the wireless sensor network, the first and the second communication protocol modules both support a first communication protocol, the third communication protocol module supports a second communication protocol, the first and the second application programming interface modules collaboratively achieve all functions of the personal area network application programming Interface (API), the all functions is a combination of the first portion functions and the second portion of functions, and the at least one terminal personal health device is connected to the application hosting device through the gateway and the wireless sensor network,
wherein when the application hosting device or the gateway transmits a message to the wireless sensor network, a transmission quality of service (QoS) control mechanism is performed by setting a transmission priority level and a positive acknowledgement mode of QoS parameters of the message.

2. The distributed application system of claim 1, wherein:
when the first application programming interface module transmits a message to the wireless sensor network through the first communication protocol module, the transmission QoS control mechanism is performed by setting the transmission priority level and the positive acknowledgement mode of the QoS parameters of the message.

3. The distributed application system of claim 1, wherein:
when the second application programming interface module transmits a message to the wireless sensor network through the second communication protocol module, the transmission QoS control mechanism is performed by setting the transmission priority level and the positive acknowledgement mode of the QoS parameters of the message.

4. The distributed application system of claim 1, wherein:
the transmission priority level of the message represents a priority order of the message to be transmitted comparing with other messages, and the positive acknowledgement mode of the message indicates whether a communication device receiving the message needs to return a positive acknowledgement packet.

5. The distributed application system of claim 2, wherein:
the application hosting device is configured to communicate with the gateway through the first application programming interface module, the first protocol communication module and the wireless sensor network; and
the first application programming interface module is configured to receive a first message generated by the personal health manage software module at an upper layer relative to the first application programming interface module in a communication protocol stack, the first application programming interface module converts the first message to a second message supporting the first communication protocol, transmits the second message to the gateway through the first communication protocol module and the wireless sensor network, wherein the first communication protocol module is at a lower layer relative to the first application programming interface module in a communication protocol stack.

6. The distributed application system of claim 5, wherein:
the gateway is configured to communicate with the application hosting device through the second application programming interface module, the second communication protocol module and the wireless sensor network; and
the second application programming interface module is configured to receive the second message transferred by the second communication protocol module at a lower layer relative to the second application programming interface module in a communication protocol stack, the second application programming interface module converts the second message to a third message supporting the second communication protocol, transmits the third message to one of the at least one terminal personal health device through the third communication protocol module at a lower layer relative to the second application programming interface module in a communication protocol stack.

7. The distributed application system of claim 3, wherein:
the gateway uses the third communication protocol module to receive a fourth message transmitted by one of the at least one terminal personal health device, the fourth message supporting the second communication protocol, the second application programming interface module converts the fourth message to a fifth message supporting the first communication protocol, and transmits the fifth message to the first communication protocol module through the second communication protocol module and the wireless sensor network.

8. The distributed application system of claim 7, wherein:
the first application programming interface module receives the fifth message received by the first communication protocol module from the wireless sensor network, converts the fifth message to a sixth message, and transfers the sixth message to the personal health manage software module.

9. The distributed application system of claim 5, wherein:
when the first application programming interface module converts the first message to the second message, the first application programming interface module firstly determines QoS attributes and the corresponding QoS control parameters while transmitting the second message to the wireless sensor network, the transmission QoS control mechanism is performed according to the QoS parameters alone or with a QoS control function of the first communication protocol module, wherein the QoS attributes are based on reliability and latency requirements and the corresponding QoS control parameters are based on transmission priority level, contention/non-contention mode and positive acknowledgment mode.

10. The distributed application system of claim 9, wherein a transmission mode of the first communication protocol module comprises a contention mode and a guaranteed timeslot mode.

11. The distributed application system of claim 10, wherein:
when the first application programming interface module uses the contention mode for transmitting the second message, the first application programming interface module also determines whether to use the positive acknowledgement mode according to the reliability parameter of the QoS attributes.

12. The distributed application system of claim 7, wherein:
when the second application programming interface module converts the fourth message to the fifth message, the second application programming interface module determines QoS attributes and the corresponding QoS control parameters while transmitting the fifth message to the wireless sensor network, the transmission QoS control mechanism is performed according to the QoS parameters alone or with a QoS control function of the second communication protocol module, wherein the QoS attributes are based on reliability and latency requirements and the corresponding QoS control parameters are based on transmission priority level, contention/non-contention mode and a positive acknowledgment mode.

13. The distributed application system of claim 12, wherein a transmission mode of the second communication protocol module comprises a contention mode and a guaranteed timeslot mode.

14. The distributed application system of claim 13, wherein:
when the second application programming interface module uses the contention mode for transmitting the fifth message, the second application programming interface module also determines whether to use the positive acknowledgement mode according to the reliability parameter of the QoS attributes.

15. The distributed application system of claim 1, wherein:
the first application programming interface module simultaneously performs segmentation and re-assembly of packets converted between the first communication protocol and the second communication protocol.

16. The distributed application system of claim 1, wherein:
the second application programming interface module simultaneously performs segmentation and re-assembly of packets converted between the first communication protocol and the second communication protocol.

17. The distributed application system of claim 5 or claim 8, wherein:
the first application programming interface module further comprises a QoS mapping table, wherein:
when the first application programming interface module determines that a new data connection is successfully established according to the first message, the second message and the fifth message, the first application programming interface module establishes a data connection record in the QoS mapping table, and configures a transmission priority level, a contention/non-contention mode and a positive acknowledgement mode of the data connection according to the new data connection; and
when the first application programming interface module determines that a data connection is successfully deleted/disconnected according to the first message, the second message and the fifth message, the first application programming interface module deletes the data connection record of the data connection in the QoS mapping table.

18. The distributed application system of claim 6 or claim 7, wherein:
the second application programming interface module further comprises a QoS mapping table, wherein:
when the second application programming interface module determines that a new data connection is successfully established according to the fourth message, the fifth message and the second message, the second application programming interface module establishes a data connection record in the QoS mapping table, and configures a transmission priority level, a contention/non-contention mode and a positive acknowledgement mode of the data connection according to the new data connection; and
when the second application programming interface module determines that a data connection is successfully deleted/disconnected according to the fourth message, the fifth message and the second message, the second application programming interface module deletes the data connection record of the data connection in the QoS mapping table.

19. The distributed application system of claim 17, wherein:
when the first application programming interface module determines it is required to transmit an non-data connection message to the wireless sensor network, the first application programming interface module directly configures a transmission priority level, a contention/non-contention mode and a positive acknowledgement mode of the non-data connection message without checking the QoS table; and
when the first application programming interface module determines it is required to transmit a data connection message to the wireless sensor network, the first application programming interface module checks a corresponding data connection record of the corresponding data connection of the data connection message in the QoS mapping table, so as to configure a transmission priority level, a contention/non-contention mode and a positive acknowledgement mode of the data connection message.

20. The distributed application system of claim 18, wherein:
when the second application programming interface module determines it is required to transmit an non-data connection message to the wireless sensor network, the second application programming interface module directly configures a transmission priority level, a contention/non-contention mode and a positive acknowledgement mode of the non-data connection message without checking the QoS table; and
when the second application programming interface module determines it is required to transmit a data connection message to the wireless sensor network, the second application programming interface module checks a corresponding data connection record of the corresponding data connection of the data connection message in the QoS mapping table, so as to configure a transmission priority level, a contention/non-contention mode and a positive acknowledgement mode of the data connection message.

21. A method for controlling quality of service in data transmission, applicable to a distributed application system comprising an application hosting device, a wireless sensor network and a gateway, the method for controlling quality of service in message transmission comprising:
the application hosting device transmitting a message to at least one terminal personal health device through the wireless sensor network and the gateway, wherein the application hosting device and the gateway both support a first communication protocol, the at least one terminal personal health device is connected to the application hosting device through the gateway, and the at least one terminal personal health device and the gateway both support a second communication protocol;
the at least one terminal personal health device transmits a message to the application hosting device through the gateway and the wireless sensor network; and
when the application hosting device or the gateway transmits a message to the wireless sensor network, the application hosting device or the gateway performs a transmission quality of service (QoS) control mechanism by setting a transmission priority level and a positive acknowledgement mode of QoS parameters of the message.

22. The method for controlling quality of service in data transmission of claim 21, wherein the transmission priority level of the message represents a priority order of the message to be transmitted comparing with other messages, and the positive acknowledgement mode of the message indicates whether a communication device receiving the message needs to return a positive acknowledgement packet.

23. The method for controlling quality of service in data transmission of claim 22, wherein before the step of setting a transmission priority level and a positive acknowledgement mode of QoS parameters of the message, the method further comprises:
the application hosting device or the gateway selects to configure a transmission priority level, a transmission mode of the first communication protocol and a positive acknowledgement mode of the message to be transmitted to the wireless sensor network according to a QoS attribute of the message to be transmitted.

24. The method for controlling quality of service in data transmission of claim 23, wherein the transmission mode of the first communication protocol comprises a contention mode and a guaranteed timeslot mode.

25. The method for controlling quality of service in data transmission of claim 24, further comprising:
when the gateway determines that a new data connection is successfully established according to content of the received message or the message to be transmitted, the gateway establishes a data connection record in a QoS mapping table, and configures a transmission priority level, a contention/non-contention mode and a positive acknowledgement mode of a data connection according to a QoS attribute of the new data connection; and
when the gateway determines that a data connection is successfully deleted/disconnected, the gateway deletes the corresponding connection record of the data connection in the QoS mapping table.

26. The method for controlling quality of service in data transmission of claim 24, further comprising:
when the application hosting device determines that a new data connection is successfully established according to content of the received message or the message to be transmitted, the application hosting device establishes a data connection record in a QoS mapping table, and configures a transmission priority level, a contention/non-contention mode and a positive acknowledgement mode of a data connection according to a QoS attribute of the new data connection; and
when the application hosting device determines that a data connection is successfully deleted/disconnected, the application hosting device deletes the corresponding connection record of the data connection in the QoS mapping table.

27. The method for controlling quality of service in data transmission of claim 25, further comprising:
when the gateway determines it is required to transmit a non-data connection message to the wireless sensor network, the gateway directly configures a transmission priority level, a contention/non-contention mode and a positive acknowledgement mode of the non-data connection message without checking the QoS mapping table.

28. The method for controlling quality of service in data transmission of claim 26, further comprising:
when the application hosting device determines it is required to transmit a non-data connection message to the wireless sensor network, the application hosting device directly configures a transmission priority level, a contention/non-contention mode and a positive acknowledgement mode of the non-data connection message without checking the QoS mapping table.

29. The method for controlling quality of service in data transmission of claim 25, further comprising:
when the gateway receives a data connection message from the personal health device, the gateway firstly checks a corresponding data connection record of a corresponding data connection in the QoS mapping table, and configures a transmission priority level, a contention/non-contention mode and a positive acknowledgement mode for transmitting the data connection message to the wireless sensor network according to the corresponding data connection record.

30. The method for controlling quality of service in data transmission of claim 26, further comprising:
when the application hosting device transmits a data connection message belonging to a data connection to the wireless sensor network, the application hosting device firstly checks a corresponding data connection record of the corresponding data connection in the QoS mapping table, and configures a transmission priority level, a contention/non-contention mode and a positive acknowledgement mode for transmitting the data connection message to the wireless sensor network according to the corresponding data connection record.
